(19) United States
(12) Patent Application Publication
(10) Pub. No.: US 2021/0398656 A1
(43) Pub. Date: Dec. 23, 2021

(54) Optimizing Treatment Administration and Management for GI Diseases

(71) Applicant: Vivante Health, Inc., Houston, TX (US)

(72) Inventor: Simon Mathews, Clarksville, MD (US)

(21) Appl. No.: 16/903,584

(22) Filed: Jun. 17, 2020

(51) Int. Cl. G16H 40/20; G16H 50/20; G16H 10/20; G16H 70/20; G16H 70/60; G16H 20/60; G16H 20/70; G16H 20/10; G16H 40/67; G06Q 10/10; G06Q 30/04; G16H 50/70; G16H 80/00; A61B 5/00; A61B 5/16; G09B 23/28

(52) U.S. Cl. CPC G16H 40/20; G09B 23/28; G16H 10/20; G16H 70/20; G16H 70/60; G16H 20/60; G16H 20/70; G16H 20/10; G16H 40/67; G06Q 10/10; G06Q 30/04; G16H 50/70; G16H 80/00; A61B 5/0022; A61B 5/4833; A61B 5/4848; A61B 5/4842; A61B 5/165; A61B 5/4211; G16H 50/20

(57) ABSTRACT

Disclosed is a system with a webserver for improving treatment of gastrointestinal diseases by use of questionnaires for the patient that are updated continuously to provide the optimal standard of care, and automatic analysis of responses to the questionnaire to provide an assessment and action plan. The action plan may include treatment or action courses, education, dietary and medication adjustments, testing, and follow-up appointments. The action plan and questionnaire can be automatically optimized by comparing results for patients following it with known treatment results.

### BACKGROUND

In treating gastrointestinal diseases, including chronic digestive diseases, such as celiac disease, Crohn's disease, ulcerative colitis, irritable bowel syndrome, there are often multiple testing and diagnostic regimens the patient undergoes before the clinician can arrive at an assessment or diagnosis. These evaluations are based on patient-completed background information, patient interview responses to the clinician's questions (which can lead to omissions in significant information), as well as more traditional laboratory and other diagnostic testing, all of such input often being gathered and reported separately. Similarly, after a diagnosis is made, further assessment and management occurs asynchronously. This frequently involves subsequent testing, medication, lifestyle, and dietary adjustments which are often enacted and monitored by members of a care team, other than the physician. These multiple and complex steps result in fragmentation of care and clinical information can lead to the patient potentially not receiving the standard of care treatment during the diagnostic and management periods.

Moreover, the more quickly and efficiently information about the patient can be gathered, and the more quickly follow-up treatment, management, or diagnostic testing appointments can be scheduled by the physician or the care team, the better the relief for the patient. Moreover, for the physician, the more quickly all services rendered can be billed for reimbursement, the more quickly the reimbursement can be received.

Enhancing progress through these steps can present unusual challenges for the IBD patient, especially if diagnostic data for the patient is being continuously sourced from the patient; e.g., where the patient is monitored for one or more of methane or hydrogen in the breath (or feces), or for levels of gut bacteria making up the microbiome. See e.g., U.S. application Ser. Nos. 16/735,734 and 16/735,427 (incorporated by reference).

Thus what is needed is a system to improve the initial patient assessment and positively identify any gaps in best practices by, e.g., comparison with a standardized (but frequently updated) patient assessment and corresponding management plan, including a plan to systematically gather data and do decision support to identify any needed steps to improve care; which will thereby provide faster implementation of treatment and/or disease management, which is also more comprehensive in scope and of superior effectiveness, and which is accomplished by implementing all aspects of best clinical practices.

### SUMMARY

The invention provides, following data and patient profile analysis, a screen display, allowing said enrolled persons, associated physicians and other partner entities data capture including a profile of enrolled patients, such as identifying data; allergies, medications, contra-indicated medications, and medical testing information. The dashboard provides the patient a detailed questionnaire relating to their gastrointestinal (“GI”) health, overall health, and treatments or other health-related actions. Patient responses are relayed to a webserver which is capable (using AI) of making a preliminary assessment of the GI health and status and/or current treatment deficiencies, and based on the preliminary assessment, recommending a primary action plan which may include further interventions, including treatment or action courses, education, dietary and medication adjustments, testing, and follow-up appointments. The webserver also invoices for reimbursement from insurance, Medicare, Medicaid or another state actor.

Exemplary patient questions in the questionnaire would relate to assessing the need for scheduling an appointment with a gastroenterologist, changing diet or changing or beginning medication, or for having an endoscopy or other test. The questionnaire is continuously reviewed and updated to cover questions relating to all aspects of best practices. Based on the patient's responses, the preliminary assessment, suggested prescriptions and diet, overall action or treatment plan including supplying patient educational materials, and the appointments for the specialist or testing can be accepted on the dashboard by the physician or caregiver. Appointments may be automatically scheduled if desired.

The responses of the patient control whether and what type of educational materials are sent to the patient. Patients with a preliminary diagnosis of gastroesophageal reflux disease (“GERD”) would receive disease-appropriate education materials, including dietary advice, for example, avoiding chocolate, peppermint, fried or fatty foods, coffee, or alcoholic beverages, which are known to trigger reflux and heartburn.

The webserver and dashboard can be controlled by the physician or a third party. If the webserver is controlled by a third party, then patient confidentiality and all HIPPA requirements must be observed, so patients would preferably be anonymous in the system. In such case, the physician would preferably be under contract with the webserver host entity to enact any suggested appointments or testing, or any part of a suggested action plan or treatment protocol.

All interactions between the patient and the webserver, including the questionnaire, can be conducted, on a mobile app, on the patient's mobile phone or device, or on a computer or other internet access device. Any type of telemedicine communication through a wireless communicable link between the patient and webserver, or physician, can be utilized.

The system can be applied as a treatment protocol by adding steps of monitoring patients health status, or health status indicators, and determining if patients using the system show more improvement or faster improvement than patients outside the system. Improvement or speed of improvement is preferably determined to statistical significance, and where such positive results are observed with particular questionnaire responses or management plans, the questionnaire and/or the management plans suggested by the system are changed to those associated with positive results. Thus, the invention provides a constantly improving treatment system for the patients.

### BRIEF DESCRIPTION OF THE DRAWINGS

FIG. 1 illustrates a communication network to implement the system of the invention. The optional managing entity can be the user or a caregiver, with intervention rights.

### DETAILED DESCRIPTION

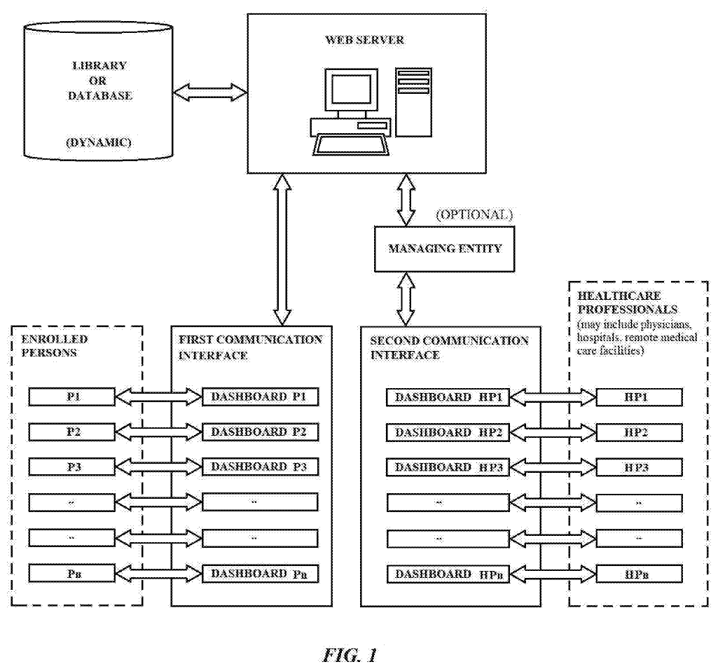

Referring to FIG. 1, the first steps undertaken from the dashboard is to administer the patient a questionnaire coving a number of subject areas pertinent to GI health. With the responses to the questionnaire, the health professional (or user) can make a preliminary patient assessment and an assessment of any deficiencies in care. The questionnaire responses also allow assessment of the patient's health maintenance status, which includes determination of (i) disease state; (ii) symptomology without disease; or (iii) individuals without symptomology or disease but who wish to optimize their GI health. Based on the assessments, the webserver devises and displays on the dashboard an action plan or treatment plan, which may include testing steps such as colorectal screening. The health professional has the option to accept, reject or modify any part or all of the preliminary assessment or action plan.

In one embodiment, an assessment inquiry module assigns a value or “weight” to the user responses to the interactive sequence of two or more linked inquiries. For example, all questionnaire topics, e.g., demographics; prior diagnosis; prior testing; prior and current symptoms; risk factors; disease severity and background information; medications; medication administration; medication adherence; lifestyle and diet triggers and modifications; quality of life assessment; psychosocial assessment; immediate health concerns; need for added testing; health maintenance status; and, mood, are inherently linked in the sense of assessing the risk of advancing GI disease and the risk of complications. The module assigns them different weights, which can be adjusted by the user, to determine a preliminary assessment or diagnosis and suggested action or treatment plan, based on the highest likelihood of a number of potential assessments and corresponding actions: ranging from no disease to severe, and from no action needed, to needing urgent medical evaluation or treatment.

In one embodiment, the dashboard can provide (a) a health management screen display, allowing patients, associated physicians, health care professionals and other partner entities to data capture including: i. enrolled patient identifying data; ii. allergies associated with said enrolled patient; iii. medications associated with said enrolled patient; iv. medications not to be prescribed with said enrolled patient; vs. other personal information entered and viewable only by said enrolled patient; and (b) a library of preventive medical programs and wellness programs are provided in assisting in the prevention of the recurrence of said enrolled patient medical condition; and (c) a person to person follow-up reminder communication system between the health care professionals and any enrolled patient having received any abnormal test results or if needed based on the assessment, is implemented and continues; until said abnormal test results are converted back to acceptable results or the needed action based on the assessment is completed or resolved.

The system may interconnect health care professionals, physicians' offices, hospitals, remote healthcare facilities and/or patient-accessible locations. The system may allow health care professionals and patients to provide clinical and scheduling information to remote healthcare providers, synchronize databases between remote locations, and provide feedback on scheduled appointments and upcoming or completed procedures.

The system also preferably allows the user or healthcare provider the ability to schedule an appointment, based on and after receiving: a patient's requested appointment from an appointment scheduler and an initial visit type from the appointment scheduler corresponding to the requested appointment; whereby, the user or provider can schedule the requested appointment when it is determined that the requested appointment corresponds to a basic appointment; and further, providing the appointment scheduler with a set of decision support tools and ability to change the requested appointment, when it is determined that the requested appointment corresponds to a complex appointment (such as analyzing test results).

A number of features and steps followed in the system are set forth in the flow chart below:

Flow Chart I: Steps for Optimizing Treatment Administration and Management for GI Diseases:

1) Step-1: From a webserver, share with an enrolled person, a questionnaire covering e.g., necessary personal details, details of medical history, current health condition, details of emergency (if any) and optionally, requested appointment schedule.
The questionnaire is shared with a corresponding dashboard included in a first communication interface (FIG. 1).
Details of medical history may include prior test results, diagnosis, demographics, risk factors, allergies, disease severity and background information, records of gastrointestinal health, medication administration and adherence, known lifestyle and diet triggers and modifications, quality of life assessment, psychosocial assessment, and prior treatments and action plans.
Details of current health condition may include current assessment of any subject in “medical history,” as well as current mood, gastrointestinal health, medical testing/diagnosis and ongoing treatment information.

2) Step-2: Share the questionnaire responses with:

i) the Webserver, which compares the responses using AI to recommended steps as determined from a dynamic Knowledge-Database (KD), that can access a smart e-library to gain access to best clinical practices and other GI-disease treatment and diagnosis information; and

ii) a designated healthcare professional (may be a physician).

3) Step-3: Based on the questionnaire responses, Webserver uses “Artificial Intelligence” (AI) and the dynamic KD for generation of:
Preliminary assessment of disease or symptom severity; preliminary assessment of evidence based gaps in quality of care; preliminary assessment of health maintenance status; preliminary diagnosis [not always performed at this stage]; any above which the user (health professional) can accept or reject; and, where accepted by the user:
Webserver generates a Primary-Action-Plan (PAP), including: educational information about disease or symptoms; adjustment recommendations to medication or administration of medication; scheduling or contacting a medical professional for evaluation including a primary physician and/or gastroenterologist; scheduling or contacting a medical professional for the purposes of a procedure or additional test; adjustment of lifestyle and dietary habits; coordination of health maintenance activities such as test recommendations, accessing test results and diagnosis reports, and reviewing available schedules for appointments (along with follow-up routines/proposals); and requesting reimbursement for initial and other visits; and optionally automatically requesting reimbursement for all care team follow-up.

4) Step-4: Share generated PAP with designated healthcare professional, through a corresponding dashboard included in a second communication interface (FIG. 1).

5) Step-5: Based on the shared questionnaire responses and updates, as well as updated information from the KD, Webserver periodically generates an Optimized-Action-Plan (OAP), including optimized recommendations in PAP with added steps and actions and confirmation of appointments and testing schedules.

6) Step-6: Share the OAP with the Webserver through corresponding dashboard (and the second communication interface, FIG. 1).

7) Step-7: From the Webserver, share relevant portions of OAP (along confirmations/rejections/rescheduling of appointments included in it) with corresponding patients.

8) Step-8: AI can access the dynamic KD and revise the patient questionnaire and OAP in view of updated best practices information from KD.

9) Step-9: if patients using the system show more improvement or faster improvement than patients outside the system with particular questionnaire responses or OAPs, the questionnaire and/or the OAPs suggested by the Webserver are changed to those associated with positive results.

10) Step-10: At the Webserver, generate reimbursement statements and share it with respective healthcare professionals.

11) Optional Step-11: For all emergencies, appointments can be scheduled with priority determined by the Webserver on receiving a request from an enrolled person.

Because the system is designed to improve the initial patient assessment and positively identify any deviations from best practices by, e.g., by comparison with a standardized (but frequently updated) patient assessment and corresponding OAP, to thereby identify any needed steps to improve care and to more quickly implement disease management, which is also more comprehensive in scope and of superior effectiveness, the system also provides for tracking of patient symptoms, complications, adverse events and other indictors of patient status on an anonymous basis. The tracked data on patient status can be compared with patient results in literature or with patients who receive care in another setting, to determine to statistical significance, whether patient health status is improved or improved sooner for patients in the system. The comparison and either verification or refutation of the effectiveness of the system in providing more positive results or providing positive results faster, can be used to review and improve any or all aspects of the system, with a view to providing more positive results or providing positive results faster.

The null hypotheses for determining whether the system provides more positive results or positive results faster is that the patients' status either (i) does not improve or (ii) does not improve faster, as compared with other patients in literature of those receiving care elsewhere. The alternate hypothesis for (i) is status for patients in the system improved, or one or more particular disease indicators improved, and for (ii), it is that patients in the system improved faster, or one or more particular disease indicators improved faster. The patient status and particular disease indicators are the mean values from a set of patients. The null and alternate hypothesis are then subjected to a selection technique, which may be either Akaike information criterion or Bayes factor or another well-known selection technique.

If any of the means above from a patient population show a normal distribution, or where the sample size n is large (for which the means will follow a normal distribution by the Central Limit Theorem), and if the standard deviations for any of the parameters noted above are known, the z-test is appropriate, where the test statistic is defined as

$$z = \frac{\overline{x} - {μ}_{0}}{σ \text{/} \sqrt{n}}$$

Where x is the mean of the parameters; μ₀ is the predicted mean; σ is the standard deviation; and n is the number of samples (which can be the patient number or the number of the particular indicator under review).

If one or both null hypotheses are rejected at a statistically significant (p≤0.05) level (where “p” is the probability that a value at least as extreme as the test statistic would be observed under the null hypothesis) then the system is verified as effective, and changes to the questionnaire, assessments or management plans are not indicated. If one or both null hypotheses are not rejected, then all phases of the questionnaire, the assessments and the management plans would be reviewed and changes would be implemented to attempt to improve the overall patient status, the speed of improvement in the status, or to improve any indicator or the speed of improvement in any indicator.

The major steps to perform such a continuously improved treatment are set forth in the flow chart below:

Flow Chart II: Continuously Improved Treatment Method for GI Disease.

a. Compare Positive Results for Patients Using System vs. Other Patients. “Positive Results” include: (i) symptomatic amelioration or reduced symptomatic frequency; (ii) reduced interval from patient introduction to symptomatic amelioration; (iii) amelioration or reduced frequency of occurrence of any indicator; (iv) reduced interval from patient introduction to indicator amelioration;

b. If Positive Results for Patients Using System Are Not Significantly More Likely Than for Other Patients, Adjust: Questionnaire or Assessments and/or Patient Management Plans (“New Conditions or Plans”);

c. Compare Positive Results for Patients Using System vs. Other Patients under New Conditions or Plans;

d. If Positive Results for Patients Using System Are Not Significantly More Likely Than for Other Patients, Repeat steps b and c; and

e. If Positive Results for Patients Using System Are Significantly More Likely Than for Other Patients, Modify the System Questionnaire, Assessments and/or Patient Management Plans to Conform to New Conditions or Plans.

Set forth below is an algorithm which further specifies steps and actions associated with implementing the system.

Algorithm I:

Algorithm based on questionnaire responses, where the algorithm assesses risk of developing Barrett's esophagus:

[(GERD duration 5 to 10) OR (GERD duration over 10)] AND [(Never Endoscopy) OR (Endoscopy in last year) OR (Endoscopy >1 year)] AND (Age 50 or over) OR (Male) OR (Caucasian/White) OR (Hiatal hernia present) OR (Obese) OR (nocturnal GERD present) OR (Current smoker) OR (Former smoker) OR (Current other tobacco) OR (Former other tobacco) OR (positive family Barretts)

Algorithm based on questionnaire responses, where the algorithm assesses need for scheduling an urgent endoscopy because of presence of high risk clinical variables:

[[(Age 60 or over) AND (positive for dyspepsia)] OR (positive for blood) OR (positive fe anemia) OR (positive lack of appetite) OR (positive weight loss) OR (positive dysphagia) OR (positive odynophagia) OR (positive vomiting) OR positive family hx ca) OR (positive imaging abn)] AND [(Never Endoscopy) OR (Endoscopy in last year) OR (Endoscopy >1 year)]

Below are some added steps, actions and algorithms associated with establishing primary and optimized action plans (PAP and OAP).

Flow Chart III: Exemplary Assessments and Management Plans:

Characterizing your GERD

Based on your responses, individuals with similar answers may be considered to have:
(mild GERD) OR (Moderate or severe GERD)
(intermittent) OR (frequent)

complicated by: (esophagitis present) and (Barrett's esophagus present)

Management Plan: Targeted GERD education

Potential Risks

Based on your responses, individuals with similar answers may be considered to have increased risk of:

(i) Developing Barrett's Esophagus [(GERD duration 5 to 10) OR (GERD duration over 10)] AND [(Never Endoscopy) OR (Endoscopy in last year) OR (Endoscopy >1 year)] AND (Age 50 or over) OR (Male) OR (Caucasian/White) OR (Hiatal hernia present) OR (Obese) OR (nocturnal GERD present) OR (Current smoker) OR (Former smoker) OR (Current other tobacco) OR (Former other tobacco) OR (positive family Barretts)

Management Plan: Targeted GERD education Care team to make referral to gastroenterologist (ii) Needing an upper endoscopy which is a procedure where a flexible camera is inserted in the mouth to examine your GI tract [(Age 60 or over) AND (positive for dyspepsia)] OR (positive for blood) OR (positive Fe anemia) OR (positive lack of appetite) OR (positive weight loss) OR (positive dysphagia) OR (positive odynophagia) OR (positive vomiting) OR positive family hx ca) OR (positive imaging)] AND [(Never Endoscopy) OR (Endoscopy in last year) OR (Endoscopy >1 year)]

Management Plan: Targeted GERD education Care team to make referral to gastroenterologist Lifestyle Modification Optimization

Based on your responses, individuals with similar answers may see symptoms improve by:

(i) Identifying and eliminating the foods and drinks that trigger symptoms (Not confident trigger id) OR (somewhat confident trigger id)

Management Plan: Targeted GERD education; Direct user to log food and track symptoms; Care team to provide targeted GERD nutrition counseling

(ii) Raising the head of the bed (No HOB elevation) AND (nocturnal GERD present)

Management Plan: Targeted GERD education; Care team to provide targeted GERD nutrition counseling

(iii) Waiting at least 2-3 hours after eating before going to bed (No Wait before bed) OR (wait 30-60 min)

Management Plan: Targeted GERD education; Care team to provide targeted GERD nutrition counseling

(iv) Losing weight (Overweight)

Management Plan: Targeted GERD education; Direct user to log food and track symptoms; Care team to provide targeted GERD nutrition counseling

(v) Improving how consistently medications are taken (On medication) AND (Med not adherent low) OR (Med not adherent high)

Management Plan: Targeted GERD education; Care team to provide targeted GERD nutrition counseling

(vi) Taking a PPI medication the best possible way (on PPI medication) AND (Taking PPI incorrect PRN) OR (Taking PPI incorrect Time of Day)

Management Plan: Targeted GERD education; Care team to provide targeted GERD nutrition counseling

(vii) Discussing with the doctor whether to stop Zantac (On Medication) AND (on Zantac not aware)

Management Plan: Targeted GERD education; Care team to provide targeted GERD nutrition counseling

(viii) Taking a PPI medication consistently for an existing diagnosis of Barrett's esophagus [(no medication) AND (Barrett's esophagus present)] OR [(Barrett's esophagus present) AND ((on low dose H2Blocker) OR (on standard dose H2Blocker)] OR [(Barrett's esophagus present) AND ((med not adherent low) OR (med not adherent high))]

Management Plan: Targeted GERD education; Care team to provide targeted GERD nutrition counseling

Specialist Consultation

Based on your responses, individuals with similar answers may benefit from consulting with a gastroenterologist

(i) Due to a diagnosis of Barrett's esophagus (No gastroenterologist) AND (Barrett's esophagus present)

Management Plan: Targeted GERD education; Care team to make referral to gastroenterologist

(ii) Due to moderate to severe symptoms despite taking medications appropriately (No gastroenterologist) AND (on standard dose PPI) AND (Taking PPI correct Time of Day) AND (Med adherent)

Management Plan: Targeted GERD education; Care team to make referral to gastroenterologist

(iii) Due to (Needing an upper endoscopy which is a procedure where a flexible camera is inserted in the mouth to examine your GI tract)

Management Plan: Targeted GERD education; Care team to make referral to gastroenterologist

(iv) Due to risk of (Developing Barrett's Esophagus)

Management Plan: Targeted GERD education; Care team to make referral to gastroenterologist

(v) Due to need for potential additional testing (No gastroenterologist) AND (on standard dose PPI) AND (Taking PPI correct Time of Day) AND (Med adherent)

Management Plan: Targeted GERD education; Care team to make referral to gastroenterologist

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A system for continuously improving assessments and treatments for gastrointestinal conditions, comprising:
a) a webserver which analyzes responses by patients to a questionnaire relating to their gastrointestinal condition, wherein the questionnaire is continuously updated by the webserver accessing a database including information about treating gastrointestinal conditions; and wherein the webserver generates an initial assessment and an initial action plan for the patients' care based on the patient responses and displays the initial action plan for the patients for a system user;
b) and wherein the system also tracks the patients' disease indicators including symptoms, complications, adverse events and other indicators of the patients' status and compares the mean of tracked indicators of patients' status with the mean of the same indicators for other patients who did not follow the initial action plan, to determine to statistical significance, whether the patients' status is improved or improved sooner by following the initial action plan compared with the other patients, wherein the null hypotheses in the determination are that following the initial action plan did not improve the patients' status or did not improve the patients' status sooner than the other patients, and wherein if the null hypotheses are not rejected at a statistically significant (p≤0.05) level by a selection technique, the questionnaire and/or the initial action plan are modified;
c) if the questionnaire and/or the initial action plan are modified in step b), step b) is then repeated for the patients under the modified questionnaire and/or the initial action plan until the null hypotheses are rejected at a statistically significant (p≤0.05) level by a selection technique; and
d) wherein the webserver schedules patient appointments both for testing and with a care-giver, and displays them for the user, and thereafter, the webserver automatically both requests reimbursement for appointments and receives funds from the reimbursing entity.

2. The system of claim 1 wherein the webserver displays the initial assessment and action plan.

3. The system of claim 1 health professional can modify the initial assessment or action plan.

4. The system of claim 1 wherein the questionnaire includes queries about demographics; prior diagnosis; prior testing; prior and current symptoms; risk factors; disease severity; medications; medication administration; medication adherence; lifestyle and diet triggers and modifications; quality of life; psychosocial status; immediate health concerns; need for added testing; health maintenance status; and, mood.

5. The system of claim 4 wherein responses to some of the queries on the questionnaire are assigned greater weight in making the initial assessment.

6. The system of claim 1 wherein the webserver allows the user or healthcare provider to schedule a patient appointment.

7. The system of claim 6 wherein the webserver permits the user or healthcare provider to schedule a patient appointment based on a patient's requested appointment schedule.

8. The system of claim 6 wherein the webserver provides an appointment scheduler with ability to change the requested appointment, when it is determined that the requested appointment corresponds to a complex appointment.

9. The system of claim 1 wherein the action plan includes educational information about disease or symptoms; adjustment recommendations to medications or administration of medications; scheduling an appointment with a medical professional; adjustment of lifestyle and dietary habits; coordination of health maintenance activities such as test recommendations, and accessing test results and diagnosis reports.

10. The system of claim 9 wherein the action plan includes scheduling an appointment with a primary physician or gastroenterologist.

11. (canceled)

12. The system of claim 1 wherein the webserver automatically both requests reimbursement for patient follow-up by a care team and receives funds from the reimbursing entity.

13. The system of claim 1 wherein interactions between the webserver, care-giver and system user are performed on the care-giver and/or the user's mobile phone or device, or on a computer or other internet access device or telemedicine platform.

14. A system for improving treatment of patients with gastrointestinal conditions, comprising:
a webserver which analyzes responses by patients to a questionnaire relating to their gastrointestinal condition, wherein the questionnaire is continuously updated by the webserver accessing a database including information about treating gastrointestinal conditions, and wherein the webserver generates an initial assessment and an action plan based on the patient responses and displays the initial action plan for the user, and wherein the action plan is implemented by a care-giver;
positive and negative effects on the patient's gastrointestinal conditions are compared by the webserver with known treatment results, for untreated patients or patients following other protocols; and wherein if positive results for a patient following the action plan are not statistically significantly more likely than known treatment results, the webserver changes the questionnaire or action plan and the patient is again assessed for positive results following changes to the questionnaire or action plan; and
wherein the webserver schedules patient appointments both for testing and with a care-giver, and displays them for the user, and thereafter, the webserver automatically both requests reimbursement for appointments and receives funds from the reimbursing entity.

15. The system of claim 14 further including the steps of comparing the patient's positive results to the known treatment results, after the patient follows the changed questionnaire or action plan.

16. The system of claim 15 further including changing the questionnaire or action plan again if positive results for the patient following the changed action plan are not significantly more likely than known treatment results.

17. The system of claim 14 wherein positive results include: (i) symptomatic amelioration or reduced symptomatic frequency; (ii) reduced interval from patient introduction to symptomatic amelioration; (iii) amelioration or reduced frequency of occurrence of any gastrointestinal condition indicator; or (iv) reduced interval from patient introduction to gastrointestinal indicator amelioration.

18. The system of claim 14 wherein positive results for the patient vs. known treatment results reach statistical significance.

19. The system of claim 14 wherein interactions between the webserver, patient, care-giver and system user are performed on the patient's, the care-giver and/or the user's mobile phone or device, or on a computer or other internet access device or telemedicine platform.
